(19) United States
(12) Patent Application Publication
(10) Pub. No.: US 2019/0282411 A1
(43) Pub. Date: Sep. 19, 2019

(54) MENSTRUAL COLLECTION PRODUCT

(71) Applicant: Benton Biomedical, LLC, Scottsdale, AZ (US)

(72) Inventor: Danielle Rodriquez, Scottsdale, AZ (US)

(21) Appl. No.: 16/353,892

(22) Filed: Mar. 14, 2019

Related U.S. Application Data
(60) Provisional application No. 62/643,352, filed on Mar. 15, 2018.

(51) Int. Cl. A61F 13/34; A61F 13/20

(52) U.S. Cl. CPC A61F 13/34; A61F 13/26; A61F 13/2022; A61F 13/2051

(57) ABSTRACT

A menstrual collection product that includes an absorbent material layer and a flexible elongated component. The absorbent material layer has a diameter that is larger than a thickness thereof. The absorbent material layer has a generally planar configuration. The flexible elongated component is attached to and extends from the absorbent material layer.

### REFERENCE TO RELATED APPLICATION

This application claims priority to Provisional Applic. No. 62/643,352, filed on Mar. 15, 2018, the contents of which are incorporated herein by reference.

### FIELD OF THE INVENTION

The invention relates to a menstrual collection product. More particularly, the invention relates to an enhanced absorbent material menstrual collection product.

### BACKGROUND OF THE INVENTION

The female menstrual cycle has been around since humankind itself existed. Traditionally, there are two primary options for menstrual collection: a pad and a tampon. The pad does the job but most women would rather not deal with the all-day soaking and odor. The tampon also does the job but it's rather uncomfortable and can't be worn during sex.

Given these facts, women have forever talked about the need for alternatives especially in this progressive generation. A further need for a modern and advanced option exists.

### SUMMARY OF THE INVENTION

An embodiment of the invention is directed to a menstrual collection product that includes an absorbent material layer and a flexible elongated component. The absorbent material layer has a diameter that is larger than a thickness thereof. The absorbent material layer has a generally planar configuration. The flexible elongated component is attached to and extends from the absorbent material layer.

Another embodiment of the invention is directed to a menstrual collection product that includes an absorbent material layer and a flexible elongated component. The absorbent material layer has an attachment mechanism associated therewith. The flexible elongated component is attached to and extends from the absorbent material layer. The flexible elongated component may be positioned in engagement with the attachment mechanism to reduce a distance the flexible elongated component extends from the absorbent material layer.

Still another embodiment of the invention is directed to a method of using a menstrual collection product. A menstrual collection product is provided that includes an absorbent material layer and a flexible elongated component. The absorbent material layer has a diameter that is larger than a thickness thereof. The flexible elongated component is attached to and extends from the absorbent material layer. The menstrual collection product is compressed to a size that is smaller than the diameter. The compressed menstrual collection product is inserted through a vagina and into a vaginal canal. The menstrual collection product is allowed to uncompress and frictionally engage a surface in the vaginal canal. The menstrual collection product absorbs menses. The flexible elongated component is used to pull the menstrual collection product out of the vaginal canal.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a top view of a menstrual collection product according to an embodiment of the invention.

FIG. 2 is an exploded side view of the menstrual collection product.

FIG. 3 is a top view of the menstrual collection product.

FIG. 4 is a sectional view of the menstrual collection product.

FIG. 5 is a top view of an alternative embodiment of the menstrual collection product.

FIG. 6 is a side view of an applicator for use with the menstrual collection product.

FIG. 7 is a side view of the menstrual collection product in an insertion configuration.

FIG. 8 is a side view of the menstrual collection product inserted into a vaginal canal.

FIG. 9 is a side view of the menstrual collection product in a generally planar configuration.

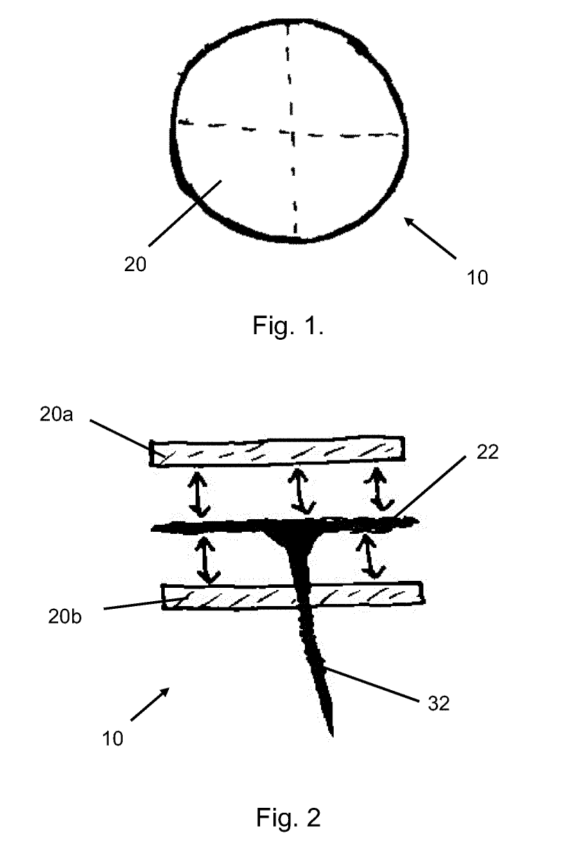

### DETAILED DESCRIPTION OF THE INVENTION

An embodiment of the invention is directed to an intra-vaginal menstrual collection product that is illustrated at 10 in the accompanying figures. An important factor in the shape of the menstrual collection product 10 is that once inserted into the vaginal canal, the menstrual collection product 10 substantially prevents menses from leaking from the vagina.

The menstrual collection product 10 generally includes an absorbent material layer 20, a reinforcing material 22 and a thread 32. The absorbent material layer 20 may be fabricated from a variety of materials using the concepts of the invention.

In certain embodiments, the absorbent material layer 20 has a generally circular shape. A person of skill in the art will appreciate that alternate shapes may be utilized with the concepts of the invention. Example of suitable alternative shapes for the absorbent material layer 20 is an oval, triangle, square, pentagon, hexagon, heptagon, octagon, nonagon and decagon.

A person of skill in the art will appreciate that there may be challenges with forming a seal between the absorbent material layer 20 and the surface of the vaginal canal when the absorbent material layer 20 has fewer sides such as triangle or square. However, the sealing ability of the absorbent material layer 20 may be enhanced in such situations by forming the menstrual collection product with a larger diameter and/or by forming the absorbent material layer 20 from a more flexible material.

Additionally, the absorbent material layer 20 may be fabricated in a variety of sizes using the concepts of the invention such that the absorbent material layer 20 may be appropriately sized for adults and adolescents. The absorbent material layer 20 should have a diameter that is larger than the diameter of the vaginal canal in which the absorbent material layer 20 is intended to be used so that frictional engagement between the absorbent material layer 20 and the wall of the vaginal canal 72 causes the absorbent material layer 20 to remain in a substantially stationary position after insertion into the vaginal canal 72. In adult humans, the width of the vaginal canal is typically between 5 centimeters and 6 centimeters.

In certain embodiments, the absorbent material layer 20 has a diameter of between about 6 centimeters and about 10 centimeters. In other embodiments, the absorbent material layer 20 has a diameter of between about 6 centimeters and about 8 centimeters.

The diameter of the absorbent material layer 20 is larger than a thickness of the absorbent material layer 20. A benefit of such a configuration is that it facilitates compressing and/or folding of the absorbent material layer 20 for insertion into the vagina 70.

In certain embodiments, a ratio of the diameter of the absorbent material layer 20 to the thickness of the absorbent material layer 20 is between about 3:1 and about 10:1. In other embodiments, the ratio of the diameter of the absorbent material layer 20 to the thickness of the absorbent material layer 20 is between about 4:1 and about 6:1. In still other embodiments, the ratio of the diameter of the absorbent material layer 20 to the thickness of the absorbent material layer 20 is about 5:1.

The absorbent material layer 20 may have a generally planar configuration such as illustrated in FIG. 4. Forming the absorbent material layer 20 with the generally planar configuration causes the absorbent material layer 20 to return to the generally planar configuration after the menstrual collection product 10 is inserted into the vaginal canal 72 and such action enables the menstrual collection product 10 to resist movement after being inserted into the vaginal canal 72.

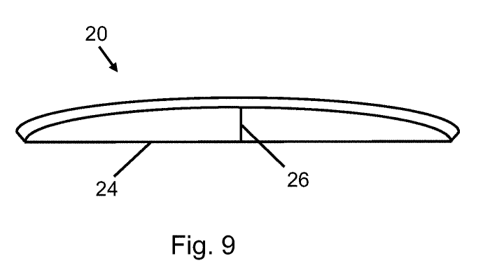

As used herein, generally planar configuration means when moving between opposite edges of the absorbent material layer 20, the absorbent material layer 20 is generally along a straight line. As used herein, generally planar configuration means that when a line 24 is drawn between opposite edges of the absorbent material layer 20, a maximum distance 26 that any portion of the absorbent material layer 20 is away from the line 24 is less than about 200 percent of the thickness of the absorbent material layer 20, as illustrated in FIG. 9.

A person of skill in the art will appreciate that the generally planar configuration also encompasses the absorbent material layer 20 having a slight convex shape (facing towards a side of the absorbent material layer 20 from which the flexible elongated member 32 extends) or a slight concave shape (facing away from a side of the absorbent material layer 20 from which the flexible elongated member 32 extends). Forming the absorbent material layer 20 with the slight convex shape may be beneficial because it could suggest to the person using the menstrual collection product 10, the direction in which the absorbent material layer 20 is to be folded prior to insertion into the vaginal canal 72.

The absorbent material layer 20 should be capable of absorbing a relatively high volume of menses and the absorbent material layer 20 should not exhibit any negative reactions when in contact with bodily tissue such as the intra-vaginal tissue during use.

In certain embodiments, the absorbent material layer 20 is an expanded foam material that is fabricated from a biologically compatible foam. One particular expanded foam that many be used in conjunction with the invention is polyurethane foam. A person of skill in the art will appreciate that it is possible to use other absorbent materials in conjunction with the concepts of the invention.

In certain embodiments, the absorbent material layer 20 includes a first layer section 20a and a second layer section 20b. Using a two-part configuration facilitates positioning the reinforcing material 22 at an intermediate location in the absorbent material layer 20 so that the reinforcing material 22 can enhance the strength of the absorbent material layer 20 without negatively impacting the absorbent characteristics of the absorbent material layer 20.

The first layer section 20a and the second layer section 20b may be formed with a thickness based upon the volume of menses that the menstrual collection product 10 is desired to collect before being removed.

In certain embodiments, the first layer section 20a and the second layer section 20b each have a similar thickness. The thickness of the first layer section 20a and the second layer section 20b may be between about 5 millimeters and about 15 millimeters. In other embodiments, the first layer section 20a and the second layer section 20b have a thickness of about 8 millimeters.

The reinforcing material 22 may have a mesh configuration. The primary forces that are experienced by the menstrual collection product 10 are during the insertion and removal of the menstrual collection product 10. While these forces are not significant, it is possible for the portions of the absorbent material layer 20 to be broken off during the insertion and removal of the menstrual collection product 10 if the reinforcing material 22 is not utilized.

The reinforcing material 22 may be forced with a shape that is similar to the shape of the absorbent material layer 20. In certain embodiments, the reinforcing material 22 has a size that is slightly smaller than the size of the absorbent material layer 20.

The reinforcing material 22 may be fabricated from a variety of materials using the concepts of the invention. The reinforcing material 22 should be fabricated from a material that does not cause any negative reactions when in contact with human tissue such as when the menstrual collection product 10 is inserted into the vagina.

The reinforcing material 22 should also resist degradation when exposed to tissue and fluids during use of the menstrual collection product 10. At a minimum, the reinforcing material 22 should not experience significant degradation during the period in which the menstrual collection product 10 is used so that the components of the menstrual collection product 10 do not separate such as when the menstrual collection product 10 is withdrawn from the vagina.

One technique for joining together the first layer section 20a, the second layer section 20b and the reinforcing material 22 is using a series of stitches 30. While it is not necessary for the stitches 30 to be placed over an entire surface of the menstrual collection product 10, the stitches 30 should be positioned in a variety of locations on the menstrual collection product 10.

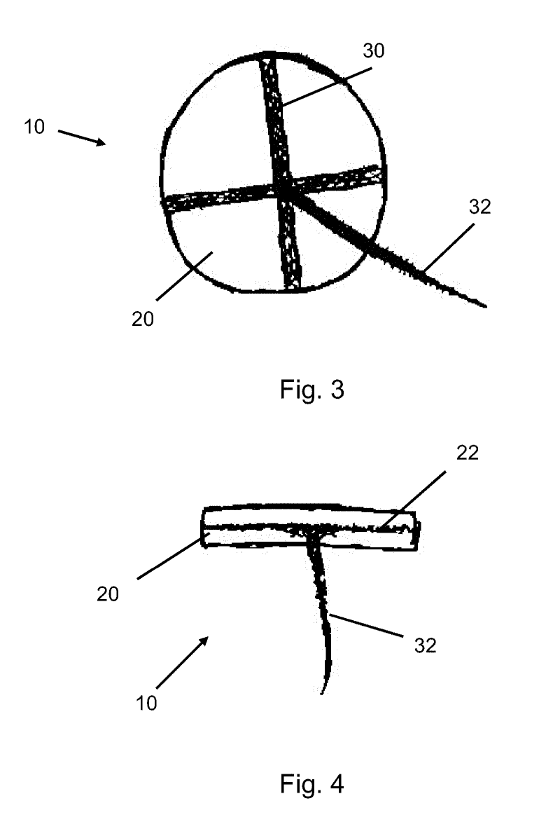

In certain embodiments, there are at least two lines of stitches 30. Each of the lines of stitches 30 may have a generally linear configuration. These lines of stitches 30 may be oriented generally transverse to each other as illustrated in FIG. 3. In other embodiments, the lines of stitches 30 extend substantially between opposite edges of the menstrual collection product 10.

Prior to forming the stitches 30, the absorbent component 20 may be at least partially compressed from an initial configuration. In certain embodiments, the absorbent component 20 is compressed at least about 50 percent from an initial configuration.

The thread utilized in forming the stitches 30 should be fabricated from a material that does not cause any negative reactions when in contact with human tissue such as when the menstrual collection product 10 is inserted into the vagina. A variety of stitching patterns may be used. An example of one suitable stitching pattern is a herringbone pattern.

The thread should also resist degradation when exposed to tissue and fluids during use of the menstrual collection product 10. At a minimum, the thread should not experience significant degradation during the period in which the menstrual collection product 10 is used so that the components of the menstrual collection product 10 do not separate such as when the menstrual collection product 10 is withdrawn from the vagina.

In certain embodiments, the menstrual collection product 10 may also include the flexible elongated component 32 extending therefrom that facilitates grasping and pulling when it is desired to remove the menstrual collection product 10 from the vagina 70. The flexible elongated component 32 thereby enhances the ability to withdraw the menstrual collection product 10.

In certain embodiments, the flexible elongated component 32 is a string. The flexible elongated component 32 may be fabricated from the same material as the thread. If the thread is used as the flexible elongated component 32, it may be desirable for a thickness of the thread to be increased such as by wrapping or tying to enhance the ability to grasp the flexible elongated component 32. In certain embodiments, the flexible elongated component 32 is joined to the reinforcing material 22.

The flexible elongated component 32 should have a length that is sufficiently long so that a distal end of the flexible elongated component 32 extends at least partially outside of the vagina when the menstrual collection product 10 is in use.

At least one of the components of the menstrual collection product 10 may have antimicrobial characteristics. These antimicrobial characteristics may be native to the component or may be added to the component during the process of fabricating the menstrual collection product 10.

At least a portion of the menstrual collection product 10 may be fabricated with a color that contrasts from the color of the tissue and/or menses where it is intended for the menstrual collection product 10 to be used. Using such a configuration facilitates identifying a location of the menstrual collection product 10 in an area of reduced visibility such as within the vaginal canal.

The first layer section 20a may be fabricated from a different color than the second layer section 20b so that a person using the menstrual collection product 10 can readily evaluate if the menstrual collection product 10 is being inserted in a correct orientation. The color of the menstrual collection product 10 may also be selected as a trademark for the entity that manufactures and/or distributes the menstrual collection product 10.

In another embodiment, the menstrual collection product 10 includes an attachment mechanism for engaging the distal end of the flexible elongated component 32 to decrease a distance that the flexible elongated component 32 extends from the other portions of the menstrual collection product 10.

The attachment mechanism may be utilized when it is desired for the distal end of the flexible elongated component 32 to not extend out of the vagina when the menstrual collection product 10 is inserted into the vagina. An example of one such instance where it may be desired for the distal end of the flexible elongated component 32 to not extend out of the vagina is when having sexual intercourse.

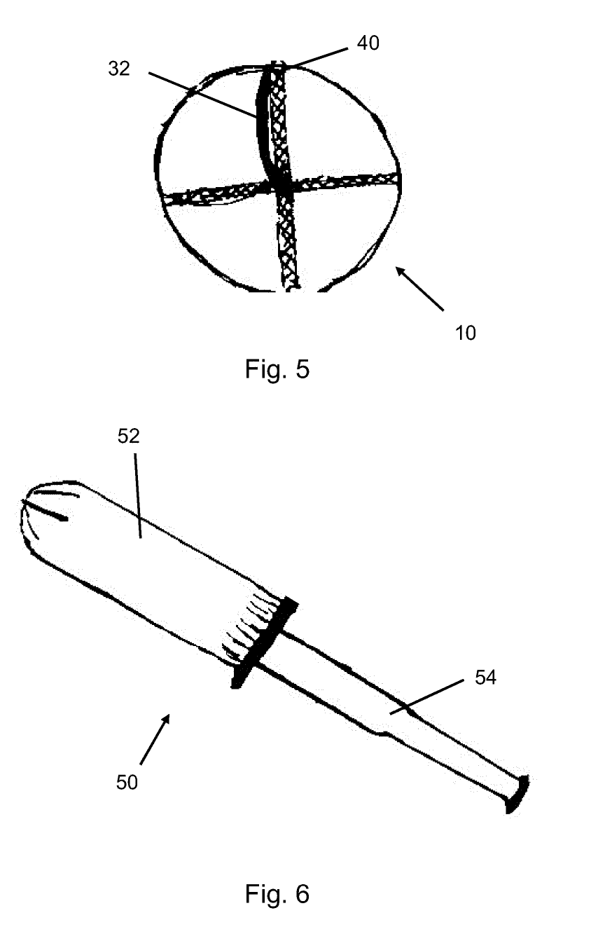

In certain embodiments, the attachment mechanism is a slit 40 formed in an edge of at least one of the absorbent material layer 20 and the reinforcing material 22, as illustrated in FIG. 5. The distal end of the flexible elongated component 32 may be placed in the slit 40 prior to insertion of the menstrual collection product 10 into the vagina.

The slit 40 extends from an outer edge of the menstrual collection product 10 toward an interior thereof. In certain embodiments, the slit 40 is oriented generally perpendicular to the location of the edge in which the slit 40 is formed.

The slit 40 is sufficiently long to facilitate receiving the flexible elongated component 32. In certain embodiments, the slit extends up to about one-quarter of a diameter of the menstrual collection product 10.

In another embodiment, the slit extends at least partially around the edge of the menstrual collection product 10 and is positioned intermediate upper and lower surfaces of the menstrual collection product 10. In other embodiments, the slit is formed in the edge and extends all the way around the menstrual collection product 10. This embodiment enables substantially all of the flexible elongated component 32 to be placed in the slit.

When it is desired to remove the menstrual collection product 10 from the vagina, an intermediate portion of the flexible elongated component is grasped to cause the distal end of the flexible elongated component 32 to be dislodged from the slit 40. The flexible elongated component 32 is then pulled to remove the menstrual collection product 10 from the vagina.

In one embodiment, the menstrual collection product 10 is manually inserted into the vagina using fingers. In another embodiment, an applicator 50 such as illustrated in FIG. 6 is used in conjunction with the menstrual collection product 10. The applicator 50 may enhance the ability to correctly insert the menstrual collection product 10 into the vagina.

The applicator 50 may generally include an outer sleeve 52 and a plunger 54. The applicator 50 may have a shape and operation that are similar to applicators that are used in conjunction with tampons.

The outer sleeve 52 may be fabricated with a generally cylindrical configuration. A bore that extends through the outer sleeve may have a length and a diameter that are sufficiently large so that the menstrual collection product 10 may be stored therein. The plunger 54 is slidable through the bore to cause the menstrual collection product 10 to be discharged from outer sleeve 52.

To enhance the ability to maintain the menstrual collection product 10 in a pathogen free and undamaged condition until it is desired to use the menstrual collection product 10, the menstrual collection product 10 may be stored in a package.

In situations where the menstrual collection product 10 is intended for a single use, the package may be a wrapper that is fabricated from a relatively thin flexible material that is torn open when it is desired to use the menstrual collection product 10. A person of skill in the art will appreciate that a variety of materials may be used to fabricate this type of package.

In other embodiments, where it is desired to reuse the menstrual collection product 10, the menstrual collection product 10 may be stored in a resealable package. The resealable package may also be used in disposing of the menstrual collection product 10 after use. The resealable package may be configured similar to a zip-top food storage bag. In other embodiments, the resealable package may be fabricated from a rigid material.

As an initial step in inserting the menstrual collection product 10 into the vagina, the menstrual collection product 10 may be folded or compressed to decrease a size thereof. In one such embodiment, the menstrual collection product 10 is folded at least two times. In other embodiments, the menstrual collection product 10 is collapsed upon itself in a non-folding manner.

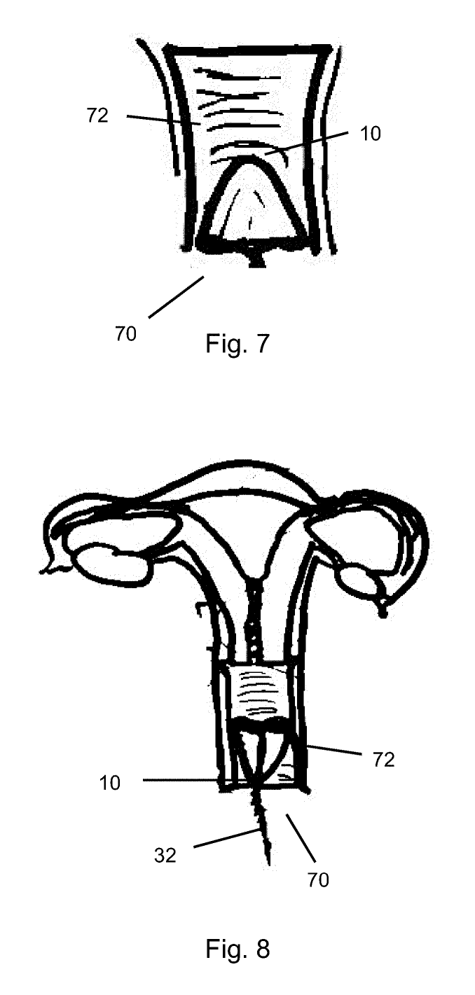

The menstrual collection product 10 is then inserted into the vagina 70 either using an applicator or fingers. FIG. 7 illustrates the menstrual collection product 10 in a partially collapsed configuration while being inserted into a vagina 70.

After insertion into the vaginal canal 72, the menstrual collection product 10 returns at least partially to a generally planar configuration as illustrated in FIG. 8 to seal the vaginal canal 72 and substantially prevent menses from leaking from the vagina 70.

The menstrual collection product 10 is situated to absorb menses discharged during menstruation. The distal end of the flexible elongated component 32 may at least partially extend from the vagina 70 so that the flexible elongated component 32 may be grasped and pulled to withdraw the menstrual collection product 10 from the vagina 70 when desired.

The menstrual collection product 10 may facilitate utilization for a longer period of time than is possible with conventional tampons because of the enhanced volume of menses that can be collected in the menstrual collection product 10 as compared to conventional tampons.

The materials used to fabricate the menstrual collection product 10 are more flexible than conventional tampons so that the menstrual collection product 10 provides enhanced comfort compared to conventional tampons.

Another benefit of the menstrual collection product 10 is that the menstrual collection product 10 may be used during sexual intercourse such as by engaging the distal end of the flexible elongated component 32 in the slit 40. The absorbent characteristics of the menstrual collection product 10 may absorb semen to reduce the potential of the semen leaking from the vagina after sexual intercourse. The menstrual collection product 10 may also incorporate a spermicide to enhance the functionality of the menstrual collection product 10.

In another embodiment, it is possible for the menstrual collection product 10 to be cleaned such as by rinsing after removal from the vagina 70. Thereafter, the menstrual collection product 10 may be reinserted into the vagina 10. This process may be repeated multiple times. In certain embodiments, the washing is performed using warm water and a vaginally-compatible soap. The menstrual collection product 10 may also be dried prior to reinsertion. Such a process not only reduces the costs as compared to conventional tampons and pads but also avoids the need for the person to carry additional tampons and/or pads.

In the preceding detailed description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The preceding detailed description, therefore, is not to be taken in a limiting sense, and the scope of the present invention is defined by the appended claims.

It is contemplated that features disclosed in this application, as well as those described in the above applications incorporated by reference, can be mixed and matched to suit particular circumstances. Various other modifications and changes will be apparent to those of ordinary skill.

1. A menstrual collection product comprising:
an absorbent material layer having a diameter that is larger than a thickness thereof, wherein the absorbent material layer has a generally planar configuration; and
a flexible elongated component that is attached to and extends from the absorbent material layer.

2. The menstrual collection product of claim 1, wherein the absorbent material layer comprises foam.

3. The menstrual collection product of claim 1, wherein the absorbent material layer comprises a first layer section, a second layer section and a reinforcing material that is between the first layer section and the second layer section.

4. The menstrual collection product of claim 3, wherein the first layer section, the second layer section and the reinforcing layer are retained in a substantially stationary position with respect to each other using a plurality of stitches through the first layer section, the second layer section and the reinforcing layer.

5. The menstrual collection product of claim 3, wherein the flexible elongated component is attached to the reinforcing material and wherein the reinforcing material has a mesh configuration.

6. The menstrual collection product of claim 1, wherein the absorbent material layer has an attachment mechanism associated therewith and wherein the flexible elongated component may be positioned in engagement with the attachment mechanism to reduce a distance the flexible elongated component extends from the absorbent material layer.

7. The menstrual collection product of claim 6, wherein the absorbent material layer comprises an outer edge, wherein the attachment mechanism comprises a slit formed in the outer edge and wherein the flexible elongated component is a string.

8. The menstrual collection product of claim 1, a ratio of a diameter of the absorbent material layer to a thickness of the absorbent material is between about 4:1 and 6:1.

9. The menstrual collection product of claim 1, where in the absorbent material layer is in a shape of at least one of circular, oval, triangle, square, pentagon, hexagon, heptagon, octagon, nonagon and decagon.

10. The menstrual collection product of claim 1, wherein at least a portion of the absorbent material layer and the flexible elongated component is fabricated with a color that contrasts from a color of menses and a color of vaginal tissue.

11. A menstrual collection product comprising:
an absorbent material layer having an attachment mechanism associated therewith; and
a flexible elongated component that is attached to and extends from the absorbent material layer, wherein the flexible elongated component may be positioned in engagement with the attachment mechanism to reduce a distance the flexible elongated component extends from the absorbent material layer.

12. The menstrual collection product of claim 11, wherein the absorbent material layer has a diameter that is larger than a thickness thereof and wherein the absorbent material layer comprises a first layer section, a second layer section and a reinforcing material that is between the first layer section and the second layer section.

13. The menstrual collection product of claim 11, wherein the absorbent material layer comprises foam and wherein a ratio of a diameter of the absorbent material layer to a thickness of the absorbent material layer is between about 4:1 and 6:1.

14. The menstrual collection product of claim 11, wherein the absorbent material layer comprises an outer edge, wherein the attachment mechanism comprises a slit formed in the outer edge and wherein the flexible elongated component is a string.

15. A method of using a menstrual collection product comprising:
providing a menstrual collection product comprising an absorbent material layer and a flexible elongated component, wherein the absorbent material layer has a diameter that is larger than a thickness thereof and wherein the flexible elongated component is attached to and extends from the absorbent material layer;
compressing the menstrual collection product to a size that is smaller than the diameter;
inserting the compressed menstrual collection product through a vagina and into a vaginal canal;
allowing the menstrual collection product to uncompress and frictionally engage a surface in the vaginal canal;
absorbing menses in the menstrual collection product; and
using the flexible elongated component to pull the menstrual collection product out of the vaginal canal.

16. The method of claim 15, and wherein the method further comprises positioning the flexible elongated member in engagement with an attachment mechanism on the absorbent material layer to reduce a distance that the flexible elongated member extends from the absorbent material layer.

17. The method of claim 16, wherein the absorbent material layer comprises an outer edge, wherein the attachment mechanism comprises a slit formed in the outer edge and further comprising disengaging the flexible elongated member from the attachment mechanism prior to using the flexible elongated member to pull the menstrual collection product from the vaginal canal.

18. The method of claim 15, wherein the absorbent material layer comprises a first layer section, a second layer section and a reinforcing material positioned between the first layer section and the second layer section and wherein the reinforcing material is attached to the flexible elongated component and wherein the reinforcing material prevents a portion of the absorbent material layer from separating from the absorbent material layer while the menstrual collection product is inserted into and removed from the vaginal canal.

19. The method of claim 18, wherein the absorbent material layer comprises foam, wherein the absorbent material layer has a generally planar configuration and wherein a ratio of a diameter of the absorbent material layer to a thickness of the absorbent material layer is between about 4:1 and 6:1.

20. The method of claim 15, wherein at least a portion of the absorbent material layer or the flexible elongated component is fabricated with a color that contrasts from a color of menses and a color of vaginal tissue and wherein the method further comprises using the contrasting color of at least the portion of the absorbent material layer or the flexible elongated component to evaluate whether the menstrual collection product was accurately inserted in the vaginal canal.
